(19) United States
(12) Patent Application Publication
(10) Pub. No.: US 2017/0112437 A1
(43) Pub. Date: Apr. 27, 2017

(54) Measurement of Hydration, Edema, and Bioelectrical Impedance

(71) Applicants: F. Frederick Dyer, Tazewell, TN (US); Jaspal S. Attrey, Charlotte, NC (US); Belinda R. Dowdy, Raleigh, NC (US)

(72) Inventors: F. Frederick Dyer, Tazewell, TN (US); Jaspal S. Attrey, Charlotte, NC (US); Belinda R. Dowdy, Raleigh, NC (US)

(21) Appl. No.: 15/331,823

(22) Filed: Oct. 22, 2016

Related U.S. Application Data
(60) Provisional application No. 62/246,804, filed on Oct. 27, 2015.

(51) Int. Cl. A61B 5/00; A61B 5/05

(52) U.S. Cl. CPC A61B 5/4875; A61B 5/05; A61B 5/7246; A61B 5/742

(57) ABSTRACT

A method of determining a degree of hydration of a sample, by generating a radio frequency signal with a frequency of no less than about two megahertz. The radio frequency signal is directed into the sample with an antenna that does not contact the surface of the sample. A reflected radio frequency signal is received from the sample and compared to the reflected radio frequency signal. Differences between the directed radio frequency signal and the reflected radio frequency signal are correlated to a degree of hydration of the sample.

### FIELD

This application claims rights and priority on prior pending U.S. provisional patent application Ser. No. 62/246,804 filed 2015 Oct. 27. This invention relates to the field of biomedical instrumentation. More particularly, this invention relates to non-invasive measurement of tissue hydration.

### INTRODUCTION

The electrical impedance of a sample is one method of determining the amount of conductive fluid, such as water, within the sample. In the medical field, bioelectrical impedance can be used to determine the hydration of a tissue sample, including in-situ living tissue, such as that of a patient.

Conventional bioelectrical impedance measurement devices operate at relatively low frequencies, such as below about two megahertz. The measurements are taken by connecting the first ends of two wire leads to the measurement device, and connecting the second ends of the two wire leads to two separate electrode pads. The pads are attached to the tissue sample to be tested, such as the surface of the skin of a patient.

The adhesion process for the pads requires the skin to be properly prepared, such as by shaving away excessive hair from the skin, cleaning the skin, and degreasing the skin to some degree, so that the pads both adhere properly and make good contact with the skin. When readings are desired at a new location on the patient (or on a different sample) the preparations must be repeated.

These necessary preparations not only increase the amount of time that is required to take such readings, but also introduce variables into the reading process, which could skew the readings from patient to patient, time to time, or care-giver to care-giver. Furthermore, these issues could possibly result, in some instances, in readings being taken less frequently than they should be, because of the amount of preparation overhead that is involved.

What is needed, therefore, is a system that reduces issues such as those described above, at least in part.

### SUMMARY

The above and other needs are met by a method of determining a degree of hydration of a sample, by generating a radio frequency signal with a frequency of no less than about two megahertz. The radio frequency signal is directed into the sample with an antenna that does not contact the surface of the sample. A reflected radio frequency signal is received from the sample and compared to the reflected radio frequency signal. Differences between the directed radio frequency signal and the reflected radio frequency signal are correlated to a degree of hydration of the sample.

In various embodiments, the frequency is no more than about three gigahertz. In some embodiments, the radio frequency signal is directed into the sample with a first antenna and the reflected radio frequency signal is received with a second antenna that is different from the first antenna. In other embodiments, the radio frequency signal is directed into the sample with a first antenna and the reflected radio frequency signal is received with the first antenna.

In some embodiments, the antenna is disposed within a probe, and the probe includes a front plate that is disposed between the antenna and the sample. In some embodiments, the front plate is electrically insulating. In some embodiments, the front plate is formed of at least one of paper, cardboard, plastic, and a semiconducting material. In some embodiments, the front plate is formed of layers of material.

In some embodiments, the antenna is disposed within a probe, and the probe includes a back plate that is disposed opposite the antenna from the sample. In some embodiments, the back plate is electrically conductive. In some embodiments, the back plate is formed of at least one of copper, aluminum, and a semiconducting material. In some embodiments, the back plate is electrically grounded to a device that generates the radio frequency signal.

In some embodiments, the radio frequency signal is generated with an oscillator. In some embodiments, the radio frequency signal is generated with a network analyzer. In some embodiments, the reflected radio frequency signal is processed with a radio frequency processor that at least one of filters and amplifies the reflected radio frequency signal.

### DRAWINGS

Further advantages of the invention are apparent by reference to the detailed description when considered in conjunction with the figures, which are not to scale so as to more clearly show the details, wherein like reference numbers indicate like elements throughout the several views, and wherein:

FIG. 1 is a functional block diagram of a two-antenna, oscillator-based hydration measurement system according to an embodiment of the present invention.

FIG. 2 is a functional block diagram of a one-antenna, oscillator-based hydration measurement system according to an embodiment of the present invention.

FIG. 3 is a functional block diagram of a two-antenna, network analyzer-based hydration measurement system according to an embodiment of the present invention.

FIG. 4 is a functional block diagram of a one-antenna, network analyzer-based hydration measurement system according to an embodiment of the present invention.

FIG. 5 is a simplified diagram of a spiral configuration of a dual-antenna according to an embodiment of the present invention.

FIG. 6 is a plot depicting frequency versus signal strength for a mildly dehydrated tissue sample as read by a hydration measurement system according to an embodiment of the present invention.

FIG. 7 is a plot depicting frequency versus signal strength for a rehydrated tissue sample as read by a hydration measurement system according to an embodiment of the present invention.

### DESCRIPTION

### Oscillator-Based System

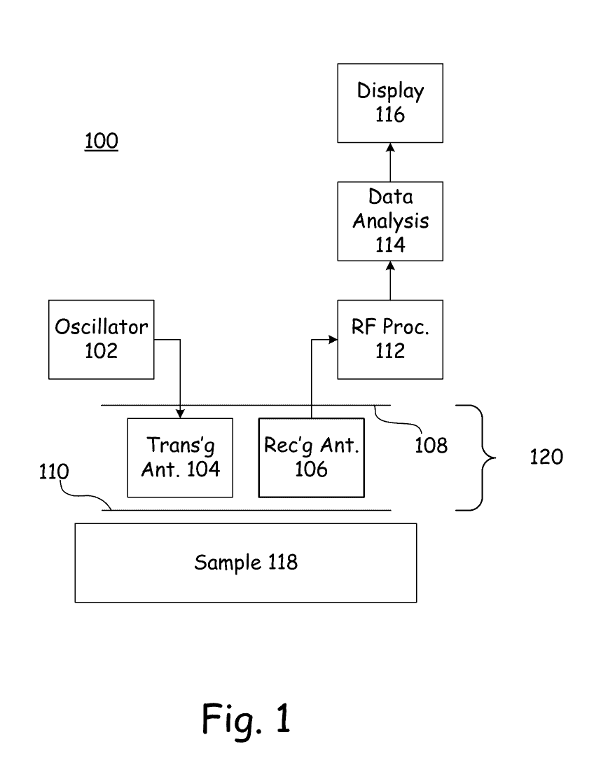

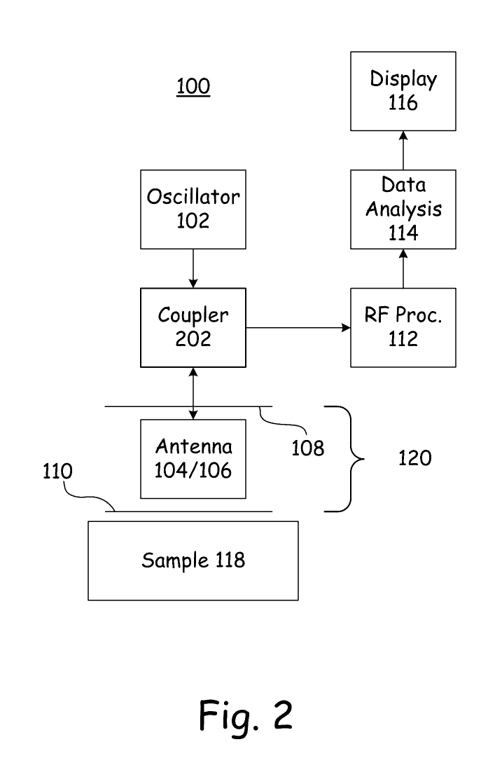

With reference now to FIGS. 1 and 2, there are depicted functional block diagrams of an oscillator-based hydration measurement system 100 according to embodiments of the present invention. FIG. 1 depicts an embodiment having two antennas 104 and 106, and FIG. 2 depicts an embodiment having one antenna 104/106, the functions of which are describe below in more detail.

In the embodiments as depicted, the radio frequency oscillator or transmitter 102 generates a radio signal. In some embodiments, the oscillator 102 produces signals within the range of from about two megahertz to about three gigahertz. In some embodiments the oscillator 102 is a Mini Circuits Model ZX95-200+, 100-200 MHz, Voltage Controlled Oscillator.

The signal is conducted to a portable or hand-held probe 120, such as through a coaxial cable. The RF signal is delivered to the sample 118 through a transmitting antenna 104. The sample 118 reflects at least a portion of a modified RF signal back to the probe 120, which receives it through a receiving antenna 106. In some embodiments, the probe 120 is placed either in physical contact with or merely proximate the sample 118, such as skin, tissue, or bodily fluid. Both antennas 104 and 106 can be placed on the same side of the sample 118, and work without making contact with the sample 118.

In the embodiment as depicted in FIG. 1, the transmitting antenna 104 and the receiving antenna 106 are separate antennas. In other embodiments, such as depicted in FIG. 2, the transmitting antenna 104 and the receiving antenna 106 are the same antenna 104/106. In some of the single antenna configurations, a directional RF coupler 202 is used to connect the probe 120 to the oscillator 102 and the RF processor 112, as depicted in FIG. 2. In this configuration, the input RF signal received by the coupler 202 from the oscillator 102 is directed to the probe 120, and the output RF signal received by the coupler 202 from the probe 120 is directed to the RF processor 112. In some embodiments the directional RF coupler 202 is a Mini Circuits Model ZFDC-20-4L, 10-1000 MHz, Directional Coupler.

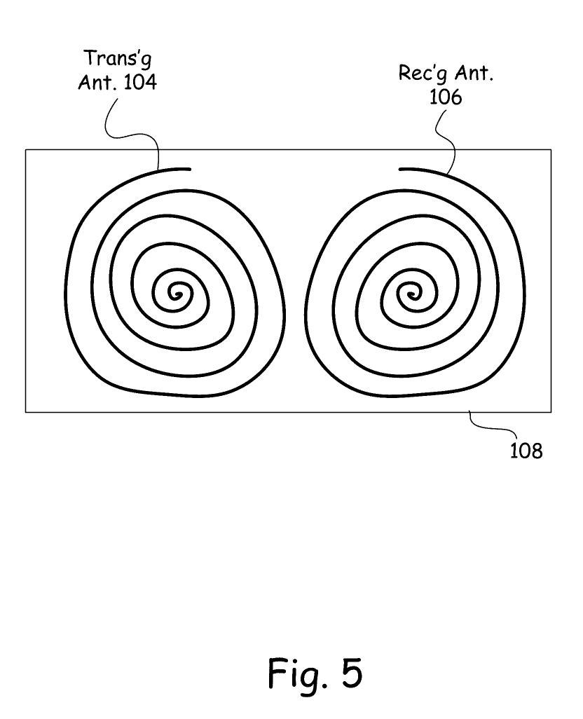

The antennas 104 and 106 are configured, in various embodiments, as spiral antennas, as represented in FIG. 5, dipole antennas, or solid conductive surface antennas. Some embodiments include a signal-reflective back plate 108 in the probe 120, such as a metal plate, film, foil, or mesh, which helps to direct the RF signal toward the sample 118. In some embodiments the back plate 108 is formed of copper or aluminum. In some embodiments the back plate 108 is formed of a semiconducting material. In some embodiments the back plate 108 is grounded to one or more of the oscillator 102, directional RF coupler 202, RF processor 112, and network analyzer 302, such as through an outer braid of a coaxial cable that is used for signal communication with the probe 120. In some embodiments the back plate 108 is separated from the antennas 104 and 106, so that it does not physically contact the antennas 104 and 106.

Some embodiments include a front plate 110 in the probe 120, such as an electrically insulating plate, which electrically isolates the antennas 104 and 106 from the surface of the sample 118, but does not significantly interfere with the transmittal or reception of the RF signals between the probe 120 and the sample 118. In some embodiments the front plate 110 is formed of paper, plastic, or cardboard. In some embodiments the front plate 110 is formed of a semiconducting material. The front plate 110 can be formed with varying thicknesses or include multiple layers.

The various embodiments described above can be used in either the dual antenna embodiments (FIG. 1) or the single antenna embodiments (FIG. 2) of the system 100, or in any of the embodiments described hereafter.

Regardless of whether a dual antenna configuration or a single antenna configuration is used, the returning signal is conducted to the radio frequency processor 112. The RF processor 112 provides functions such as filtering and amplifying of the RF signal. In some embodiments the RF processor 112 is a B & K Precision Model 2650 3 GHz Spectrum Analyzer.

The signal is then passed along to a data analysis unit 114. The data analysis unit 114 processes and interprets the RF signal, and presents information, such as text and graphics, on a display 116. In some embodiments, the data analysis unit 114 is a personal computer, or some other microprocessor-based computing device, which analyzes the signal according to the processes described hereafter.

In some embodiments the transmitted RF signal power ranges from about one milliwatt to about ten milliwatts, and the RF signal that is received from the sample 118 is a factor of from about ten to about ten thousand lower than the input signal, or in other words, from about one milliwatt to about one hundred nanowatts. In some embodiments, a higher fluid concentration in the sample 118, such as from edema or general hydration, results in a larger reflected signal amplitude, as compared to the reflected signal from tissue with less hydration or edema.

### Network Analyzer-Based System

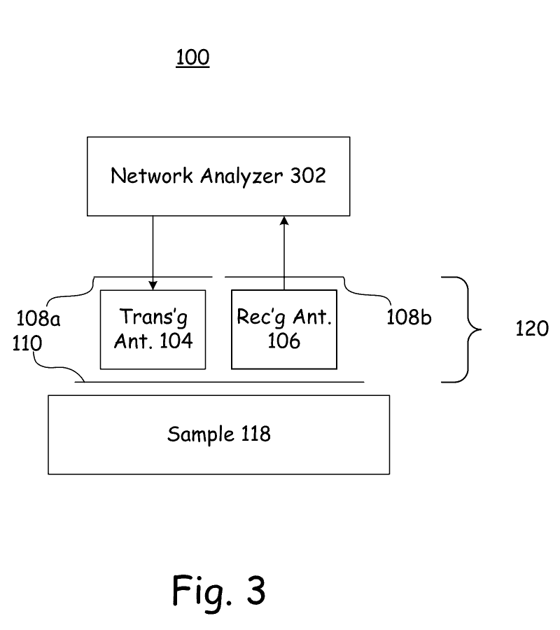

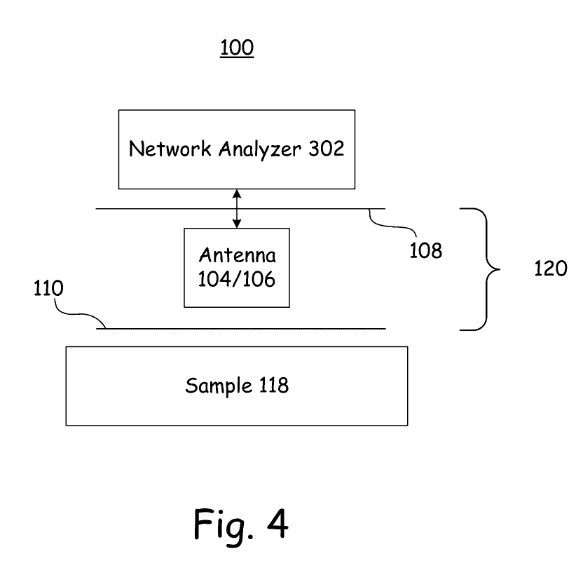

Referring now to FIGS. 3 and 4, there are depicted functional block diagrams of a network analyzer 302-based hydration measurement system 100 according to embodiments of the present invention. In these embodiments, a commercially-available vector network analyzer 302 replaces several of the components as depicted in FIGS. 1 and 2.

For example, the network analyzer 302 generates an RF signal within the range of from about two megahertz to about three gigahertz that is conducted to either a dedicated transmitting antenna 104 in a dual-antenna configuration as depicted in FIG. 3, or to a transmitting and receiving antenna 104/106 in a single-antenna configuration as depicted in FIG. 4. As before, RF energy from the antenna 104 is conducted or transmitted through the sample 118 to the antenna 106, and relayed back to the network analyzer 302, which analyzes the signal data, including frequency and transfer function (scalar and vector), and displays the information, such as on a built-in display. In some embodiments, the network analyzer is one of an HP 8753C and an NWT portable Network Analyzer.

In some embodiments the back plate 108 is separated into two back plates 108a and 108b, as depicted in FIG. 3. In these embodiments, one of the back plates 108a is adjacent to the transmitting antenna 104, and the other back plate 108b is adjacent to the receiving antenna 106. It is appreciated that these embodiments are compatible both with the network analyzer 302 embodiments, and with the oscillator 102 embodiments, as are the one-back plate 108 embodiments. In some embodiments, the back plate 108a is grounded to one of the oscillator 102 and network analyzer 302, such as through an outer braid of a coaxial cable that is used for its signal communication, and the other back plate 108b is separately grounded to one of the network analyzer 302 and RF processor 112, such as through an outer braid of the coaxial cable that is used for its signal communication.

### Applications

For measuring edema of the lower leg, for example, the probe 120 (either one antenna or two antennas) is placed between the ankle and knee and the network analyzer 302 (for example) is set to sweep between three hundred kilohertz and two hundred megahertz. Sample operating parameters include transmitted power into the sample 118 of about one milliwatts (0 dBm), and reflected power of from about −30 dBm to about −10 dBm.

Significant edema tends to be detected by the system 100 as a lower reflected power at the relatively higher frequencies under investigation. For example, about −15 dBm at about 160 MHz, instead of about −12 dBm at the same frequency for normally hydrated tissue.

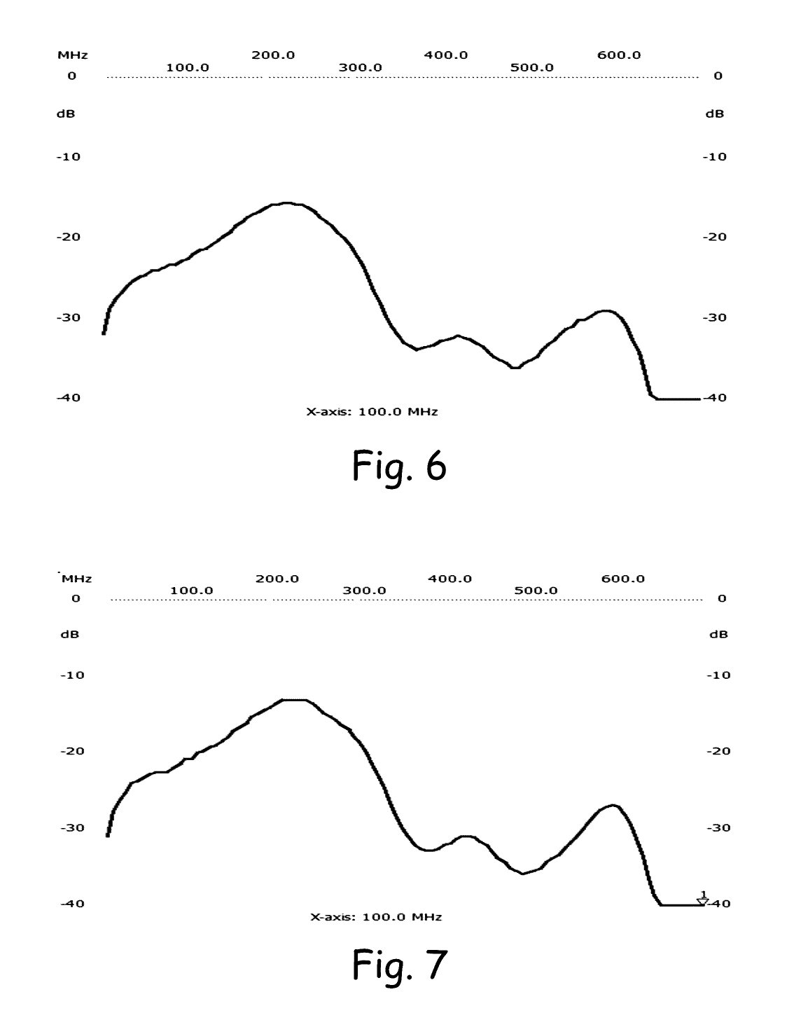

In another embodiment, the probe is placed on or near the forearm of a patient, and a frequency sweep is performed within the range of from about twenty megahertz to about 650 megahertz. FIGS. 6 and 7 show the response of forearm tissue. Three peaks are visible on these graphs. One peak that is significant for hydration is the peak at about 420 megahertz. FIG. 6 is the graph from the forearm of a patient that is relatively dehydrated, and FIG. 7 is the graph from the forearm of the same patient after some amount of hydration.

Note that the overall amplitude of the peak at about 420 megahertz has increased by about two decibels from the relatively dehydrated conditions recorded in FIG. 6 to the relatively hydrated conditions recorded in FIG. 7. For this hydration measurement, the operating parameters were as follows. The power transmitted into the tissue sample 118 was about one milliwatt (0 dBm). The reflected power was −30 dBm to −10 dBm. Particularly useful is the −33 dBm peak at about 420 megahertz.

In some embodiments, the sample 118 characteristics measured include impedance, resistance, dielectric constant, phase shift, and delay. These radio frequency electrical characteristics can be interpreted or calculated to determine multiple sample 118 properties of interest, including water content, skin conductivity, body composition, edema, lymphedema, hot flash detection, body mass index or bone density, by looking for differences in the reflected power at different frequencies.

The sample 118 does not need to be a homogeneous structure such as skin, muscle, or bone. Deeper penetration of the RF energy, with possible use of widely spaced antennas, can produce tomography data (electrical impedance tomography) that can detect organ or structural abnormalities such as collapsed lung or enlarged heart or enlarged prostate. Cancerous tumors exhibit different RF impedance properties from normal tissue, and therefore cancerous tumors could be detected by various embodiments of the present invention.

The foregoing description of embodiments for this invention has been presented for purposes of illustration and description. It is not intended to be exhaustive or to limit the invention to the precise form disclosed. Obvious modifications or variations are possible in light of the above teachings. The embodiments are chosen and described in an effort to provide illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A method of determining a degree of hydration of a sample, the method comprising the steps of:
generating a radio frequency signal with a frequency of no less than about two megahertz,
directing the radio frequency signal into the sample with an antenna that does not contact the surface of the sample,
receiving a reflected radio frequency signal from the sample,
comparing the directed radio frequency signal to the reflected radio frequency signal, and
correlating differences between the directed radio frequency signal and the reflected radio frequency signal to a degree of hydration of the sample.

2. The method of claim 1, wherein the frequency is no more than about three gigahertz.

3. The method of claim 1, wherein the radio frequency signal is directed into the sample with a first antenna and the reflected radio frequency signal is received with a second antenna that is different from the first antenna.

4. The method of claim 1, wherein the radio frequency signal is directed into the sample with a first antenna and the reflected radio frequency signal is received with the first antenna.

5. The method of claim 1, wherein the antenna is disposed within a probe, and the probe includes a front plate that is disposed between the antenna and the sample.

6. The method of claim 5, wherein the front plate is electrically insulating.

7. The method of claim 5, wherein the front plate is formed of at least one of paper, cardboard, plastic, and a semiconducting material.

8. The method of claim 5, wherein the front plate is formed of layers of material.

9. The method of claim 1, wherein the antenna is disposed within a probe, and the probe includes a back plate that is disposed opposite the antenna from the sample.

10. The method of claim 9, wherein the back plate is electrically conductive.

11. The method of claim 9, wherein the back plate is formed of at least one of copper, aluminum, and a semiconducting material.

12. The method of claim 9, wherein the back plate is electrically grounded to a device that generates the radio frequency signal.

13. The method of claim 1, wherein the radio frequency signal is generated with an oscillator.

14. The method of claim 1, wherein the radio frequency signal is generated with a network analyzer.

15. The method of claim 1, wherein the reflected radio frequency signal is processed with a radio frequency processor that at least one of filters and amplifies the reflected radio frequency signal.

16. A method of determining a degree of hydration of a sample, the method comprising the steps of:
generating a radio frequency signal with a frequency of no less than about two megahertz and no more than about three gigahertz,
directing the radio frequency signal into the sample with an antenna that does not contact the surface of the sample, wherein the antenna is disposed within a probe, and the probe includes an electrically insulating front plate that is disposed between the antenna and the sample, and an electrically conductive back plate that is disposed opposite the antenna from the sample,
receiving a reflected radio frequency signal from the sample,
comparing the directed radio frequency signal to the reflected radio frequency signal, and
correlating differences between the directed radio frequency signal and the reflected radio frequency signal to a degree of hydration of the sample.

17. The method of claim 16, wherein the radio frequency signal is directed into the sample with a first antenna and the reflected radio frequency signal is received with a second antenna that is different from the first antenna.

18. The method of claim 16, wherein the radio frequency signal is directed into the sample with a first antenna and the reflected radio frequency signal is received with the first antenna.

19. The method of claim 16, wherein the radio frequency signal is generated with one of an oscillator and a network analyzer.

20. A method of determining a degree of hydration of a sample, the method comprising the steps of:
generating a radio frequency signal with a frequency of no less than about two megahertz and no more than about three gigahertz,
directing the radio frequency signal into the sample with a first antenna that does not contact the surface of the sample, wherein the first antenna is disposed within a probe, and the probe includes an electrically insulating front plate that is disposed between the first antenna and the sample, and an electrically conductive back plate that is disposed opposite the first antenna from the sample,
receiving a reflected radio frequency signal from the sample with a second antenna that is different from the first antenna,
comparing the directed radio frequency signal to the reflected radio frequency signal, and
correlating differences between the directed radio frequency signal and the reflected radio frequency signal to a degree of hydration of the sample.
